(19) United States
(12) Patent Application Publication
(10) Pub. No.: US 2023/0032283 A1
(43) Pub. Date: Feb. 2, 2023

(54) Topical Compositions For Treating Skin Ailments

(71) Applicant: Samer Yazigi, Fulshear, TX (US)

(72) Inventor: Samer Yazigi, Fulshear, TX (US)

(21) Appl. No.: 17/853,650

(22) Filed: Jun. 29, 2022

Related U.S. Application Data
(60) Provisional application No. 63/224,101, filed on Jul. 21, 2021.

(51) Int. Cl. A61K 38/12; A61K 9/00; A61K 9/06; A61K 31/137; A61K 31/7036; A61K 31/5375; A61K 31/4174; A61K 31/65; A61K 31/245; A61K 31/47; A61K 31/085; A61K 31/27; A61K 31/755; A61K 31/045; A61K 31/05; A61K 31/125; A61K 36/14; A61P 17/10

(52) U.S. Cl. CPC A61K 38/12; A61K 9/0014; A61K 9/06; A61K 31/137; A61K 31/7036; A61K 31/5375; A61K 31/4174; A61K 31/65; A61K 31/245; A61K 31/47; A61K 31/085; A61K 31/27; A61K 31/755; A61K 31/045; A61K 31/05; A61K 31/125; A61K 36/14; A61P 17/10

(57) ABSTRACT

Topical cream or ointment medications formulated by blending admixtures of various concentrations of active ingredients with a plurality of inactive excipient ingredients for treating acne and a variety of other similar skin ailments including, folliculitis, rosacea, boil, skin-flaking, minor wounds, cuts, and adverse skin-issues related to use of covid-19 protective face-masks covered-area rashes, acne and non-lethal insect bites.

### FIELD OF INVENTION

The present invention relates to use of over-the-counter topical medications, and more particularly relates to medicated creams and ointments formulated to address and treat common inflammatory skin disorders including acne, folliculitis, rosacea, and boil. These skin disorders typically have an adverse and protracted impact on a victim's self-confidence and self-esteem. Moreover embodiments of the present invention have proven to effective for addressing skin-flaking, minor wounds, cuts, and adverse skin-issues related to use of covid-19 protective face-masks covered-area rashes, acne and non-lethal insect bites.

### BACKGROUND OF INVENTION

Common inflammatory skin disorders typified by acne, folliculitis, rosacea and boils are usually localized on one's face, forehead, chest, upper back and shoulders because of prevalence of sebaceous glands thereat. Besides inherently being uncomfortable and unsightly, such skin ailments tend to significantly adversely affect self-confidence and self-esteem.

As is well known by practitioners in the skin treatment art, for many patients undergoing conventional medical therapeutic treatment for severe acne relying upon oral doses of tetracycline or estrogen or like medications, still suffer discomfort from persisting itching and other skin-ailment symptoms attributable to experiencing minimal or essentially being non-responsive to such commonly prescribed intensive therapeutic treatments. Practitioners in the art have provided topical benzoyl peroxide for successfully treating mild to medium acne, albeit typically requiring a protracted time frame for healing of acne-related skin conditions. Practitioners in the art have also prescribed oral antibiotics for male patients and oral hormones for female patients for successfully treating more severe acne and similar adverse skin conditions, albeit typically requiring a protracted time frame for healing such adverse skin conditions.

As should be evident to practitioners skilled in the art, such protracted time frames are problematic and unacceptable for teenagers and young adults suffering from skin conditions typically manifest as facial papules and as body pustules because of unavoidable embarrassment and related debilitating and potentially traumatic effects upon adolescent development manifest as undermined self-esteem and lack of self-confidence. Thus, a shorter time frame would be critical for curing or at least assuaging conspicuous unsightly and embarrassing skin conditions suffered by adolescents especially while participating in gym or swimming or other physical fitness activities in a school or college or like social environment.

Furthermore, as evidenced during the covid-19 pandemic manifest during 2020-2022, face coverings were required or at least recommended to be worn primarily in the form of snug-fitting face-masks—as the most useful measure to prevent spreading or contracting coronavirus. Unfortunately, a prevalent unforeseen consequence of such routine face-mask wearing has caused individuals to become victims of entrapped moisture from their own breath, exacerbated by humidity and warm temperatures thereby fostering papule growth and rashes on skin disposed beneath the face-mask. Medical personnel and essential workers, ironically, have suffered virtually incessantly from this seemingly unavoidable adverse symptom on the basis of being required to wear facial coverings for protracted times, every day—not only for protecting themselves, but also for the protection of patients with whom contact is necessarily being made.

Accordingly, what is needed in the art is a convenient efficacious topical treatment comprising vasoconstrictor and antibiotic active ingredients for synergistically enabling prompt destruction of inflammation-causing bacteria by expeditiously reducing both blood supply to infected skin areas and inflammation thereof, thereby expeditiously assuaging patients' suffering from acne, folliculitis, rosacea, boils, and other skin ailments.

### SUMMARY OF INVENTION

The present invention teaches topical cream or ointment compositions comprising a synergistic formulation of vasoconstrictors and antibiotics functioning as primary active ingredients. As will be readily understood by practitioners skilled in the art, vasoconstrictors reduce inflammation and blood-supply to infected skin thereby facilitating antibiotics ingredients' ability to expeditiously kill invasive bacteria typically causing such inflammation and infection. As will hereinafter be described, a plurality of other ingredients has been found to complement and cooperate with these active ingredients to enable surprisingly effective treatment of unduly oppressive skin ailments contemplated herein. These complementary ingredients include but are not limited to anesthetics, antifungals, analgesics, steroids, and decongestants as will be elaborated and exemplified herein.

For instance, adding an active antifungal ingredient to combinations of ingredients functioning as vasoconstrictors and ingredients functioning as antibiotics have been found to effectively address and remedy fungal acne, acne vulgaris, folliculitis, and similar skin conditions without introducing any appreciable negative side-effects. Furthermore, it has also been found adding a compatible anesthetic to compositions hereof tends to relieve discomfort and even tends to attenuate pain associated with boil and other similar severe skin ailments.

As will become evident to practitioners skilled in the art, topical formulations of the present invention include a variety of antibiotic ingredients and like varieties of compatible vasoconstrictor ingredients and decongestant ingredients incorporated into a synergistic combination manifest as an ointment or a cream having a panoply of certain other ingredients characterized as being anti-fungal, anesthetic, anti-inflammatory, hydrocortisone, painkiller, and beta-blocker—for effectively treating skin inflammation and most commonly recurring other skin ailments. As will become evident to practitioners in the art, all active and inactive ingredients incorporated into formulations hereof are available over-the-counter (OTC) and have been accorded FDA-approval for OTC non-prescription medications.

As will become clear to practitioners skilled in the art, favorable outcomes have been regularly experienced by several volunteer-patients suffering from different stages of various skin ailments described and enumerated herein. These favorable outcomes were effectuated after volunteer-patients were treated with embodiments of topical formulations taught herein during which ointments or creams were regularly applied topically to particular skin disorders essentially targeting each papule or pustule and surrounding inflamed skin based upon a regimen of treatments self-administered preferably two to three times daily.

Visually indicative of the oppressive nature of skin disorders generally addressed hereunder, FIG. 1 schematically illustrates a portion of layered human skin 5 contrasting normal skin condition with abnormal skin condition. More particularly, normal healthy skin anatomy 10 is depicted comparatively with abnormal skin portion 15 afflicted with a plurality of disorders as will be elucidated hereinafter. As will be appreciated by practitioners in the art, human skin anatomy comprises layered epidermis 20, dermis 30, and hypodermis 40. Healthy normal hair follicle 50 is depicted successively passing through dermal layer 30 and epidermal layer 20, along with associated hair follicle shaft 60 projecting outwardly from epidermal layer 20. Also depicted are sweat gland 70, fat tissue 80, sebaceous gland 90—interspersed with plurality of blood vessels 100. Distinguished from such healthy hair follicle 50 there is depicted unhealthy hair follicle 55 observed to be afflicted with folliculitis characterized by skin not only being inflamed and fraught with clogged skin, but also characterized by its shaft forming a substantially rounded pimple or pustule 65 rather than forming a conventional normally linear structure 60 of healthy hair follicle 50 protruding substantially vertically from epidermal layer 20.

It is accordingly an object of the present invention to provide an over-the-counter topical ointment or cream for treating skin conditions typified by acne, folliculitis, rosacea, and skin-flaking.

It is also an object and advantage of the present invention affording a patient an over-the-counter efficacious self-administering topical cream or ointment for treating skin conditions including acne vulgaris, folliculitis, and rosacea.

It is yet another object and advantage of the present invention enabling a patient to obtain an over-the-counter ointment or cream for expeditiously being self-administered to treat and attenuate a plethora of symptoms accompanying such skin conditions as acne, folliculitis and rosacea.

These and other objects of the present invention will become apparent from the following specifications and accompanying drawing figures.

### BRIEF DESCRIPTION OF DRAWING FIGURES

FIG. 1 depicts a simplified schematic of a portion of human macro skin anatomy simultaneously illustrating both normal skin and abnormal skin.

FIG. 2 depicts an image of the upper back and adjacent shoulder area of a male 20 year-old patient suffering from persistent acne and folliculitis skin disorders thereupon.

FIG. 2A depicts the image of the persistent acne and folliculitis skin disorders depicted in FIG. 2 after being subjected to one days' thrice daily topical treatment of an embodiment of the present invention.

FIG. 3 depicts an enlarged isolated image of the upper back and adjacent shoulder area of the patient depicted in FIG. 2 after being subjected to three days' thrice daily topical treatments of an embodiment of the present invention.

FIG. 4 depicts the image of the back of the patient depicted in FIG. 2 after being subjected to five days' thrice daily topical treatments of an embodiment of the present invention.

FIG. 5 depicts an image of the face of a male 20 year-old patient suffering from mild acne skin disorder thereupon.

FIG. 6 depicts a close-up image of the face of the male patient depicted in FIG. 5.

FIG. 7 depicts the image of the face of the male patient depicted in FIG. 6 after being subjected to first 24 hours twice daily topical treatments of an embodiment of the present invention.

FIG. 8 depicts a close-up image of the face of the male patient depicted in FIG. 7 revealing corresponding implicated skin area devoid of indication of acne infection after being subjected to two days twice daily topical treatments.

FIG. 9 depicts an image of the facial left side of a male 48 year-old patient suffering from boils and folliculitis skin disorders thereupon.

FIG. 10 depicts the image of the facial left side of the patient depicted in FIG. 9 showing reduced swelling albeit with subcutaneous redness after being subjected to five days' twice daily topical treatments of an embodiment of the present invention.

FIG. 11 depicts a close-up image of the facial left side of the patient depicted in FIG. 10.

FIG. 12 depicts the image of the facial left side of the patient depicted in FIG. 9 after being subjected to eight days' twice daily topical treatments of an embodiment of the present invention.

FIG. 13 depicts an image of the skin-flaking on and around the ears of a male 48 year-old patient suffering from flaking skin disorder thereupon.

FIG. 14 depicts the image of the skin on and around the ears of the patient depicted in FIG. 13 showing only minor flaking after being subjected to six days' twice daily topical treatments of an embodiment of the present invention.

FIG. 15 depicts the image of the skin on and around the ears of the patient depicted in FIG. 13 showing ears having been cured absent any visible flaking after being subjected to eleven days' twice daily topical treatments of an embodiment of the present invention.

FIG. 16 depicts the image of the skin on and around the ears of the patient depicted in FIG. 13 showing reappearance of slight flaking more than three months after being subjected to about eleven days' twice daily topical treatments of an embodiment of the present invention.

FIG. 17 depicts an image of the facial left side face of a female teenage patient suffering from substantial acne skin disorder thereupon.

FIG. 18 depicts the image of the facial left side of the patient depicted in FIG. 17 after being subjected to one days' once daily topical treatment of an embodiment of the present invention.

FIG. 19 depicts the image of the facial left side face of the patient depicted in FIG. 17 after being subjected to three days' once daily topical treatment of an embodiment of the present invention.

FIG. 20 depicts an image of an acute rash of pustules with inflamed skin on a 19 year-old male patient's upper back and around his shoulders at beginning topical treatment of an embodiment of the present invention.

FIG. 21 depicts a close-up image of the shoulder of the patient depicted in FIG. 20 at beginning topical treatment of an embodiment of the present invention.

FIG. 22 depicts a close-up image of the upper back of the patient depicted in FIG. 20 at beginning topical treatment of an embodiment of the present invention

FIG. 23 depicts an image of the completely cleared skin on the shoulder of the patient depicted in FIG. 21 after a week being subjected to twice daily topical treatments of an embodiment of the present invention.

FIG. 24 depicts an image of the completely cleared skin on the upper back of the patient depicted in FIG. 22 after a week being subjected to twice daily topical treatments of an embodiment of the present invention.

### DETAILED DESCRIPTION OF INVENTION

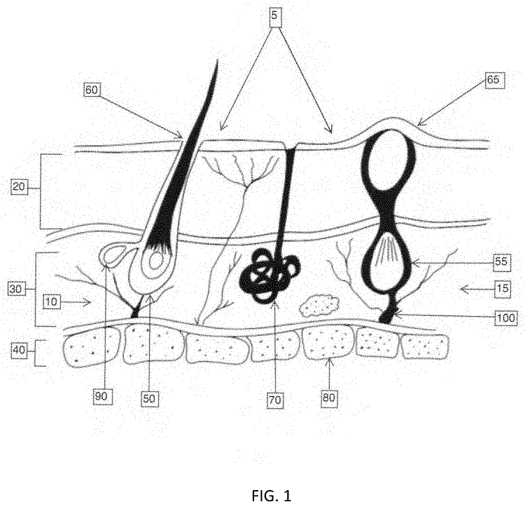

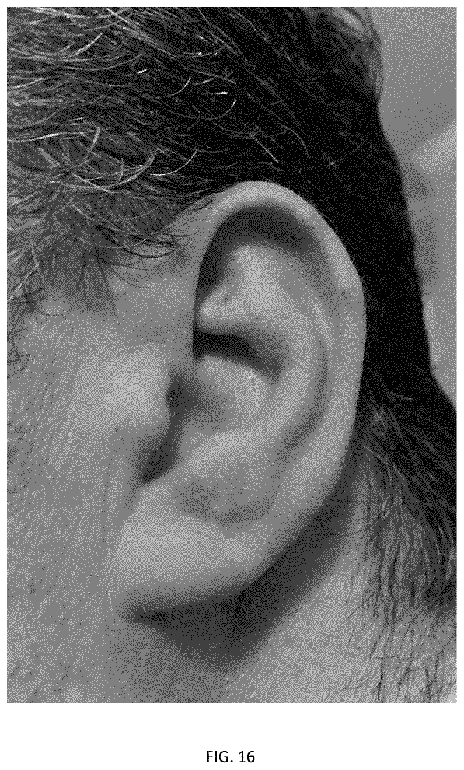

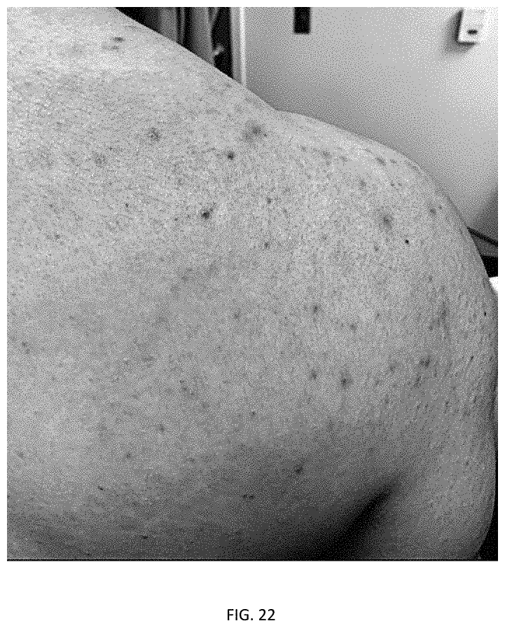

Reference is made herein to the photographic images depicted in FIGS. 1-24 which illustrate adverse symptomology manifest on several volunteer-patients' facial, ear, shoulder and back human body portions, and central face region containing the nose, cheeks, and ears; and concomitant visual attributes of skin conditions afflicting such plurality of facial, ear and back human body portions.

OTC Medications hereof comprise specially-formulated ointments or creams intended to be topically self-administered by patients to facial and bodily areas of affected skin and associated soft tissues. It has been found during several testing cycles with different patient-volunteers certain optimum outcomes have been regularly achieved by topically applying ointment or cream formulations taught herein. More particularly, such optimum outcomes regarding attenuating symptomology associated with skin disorders have been attained by preferably topically reaching—while treating essentially each papule or each pustule manifest in such adverse skin conditions, as well as simultaneously topically treating each adjacent surrounding area of inflamed skin according to a prescribed schedule—typically two to three times daily.

Notwithstanding this preferred topical treatment methodology, medicated creams or medicated ointments should only be sparingly applied to targeted inflamed and infected skin inasmuch as inadvertently affecting normal, i.e., uninfected and uninflamed areas of skin should be avoided to prevent introducing excessive antibiotic concentrations thereby potentially effectuating adverse side-effects thereupon. As will be clarified hereinafter, twice-daily appropriate doses of topical applications have been determined to typically ameliorate mild skin conditions, while at least thrice-daily appropriate doses of topical applications have been ascertained as tending to ameliorate more severe skin disorders. Indeed, empirical studies have determined the following active ingredients to be effective alone or in mixtures thereof for accomplishing the topical treatment functional objectives recited herein:

### Vasoconstrictor Ingredients

Vasoconstrictor ingredients' primarily functions are to reduce inflammation and blood supply to infected skin thereby facilitating antibiotics and like ingredients to expeditiously kill invasive bacteria. It has been found vasoconstrictor ingredients comprising preferably 0.25% by weight; notwithstanding, phenylephrine hydrochloride has been found to perform acceptably in the range of 0.125% to 1.0% by weight. Furthermore, vasoconstrictor performance contemplated hereunder has been found to be achieved by the following ingredients when admixed in a manner well known in the art with a plurality of other ingredients within certain range of concentrations: (a) ephedrine sulfate 0.1 to 1.25 percent by weight; (b) epinephrine 0.005 to 0.01 percent by weight; (c) epinephrine hydrochloride 0.005 to 0.01 percent by weight; (d) phenylephrine hydrochloride 0.25 percent by weight; and mixtures thereof.

### Antibiotic Ingredients

Antibiotic ingredients incorporated into formulations hereunder primarily function to inhibit and terminate bacterial growth on the skin. It has been observed blending into compositions hereof antibiotic ingredients comprising 500 units of bacitracin zinc were effective treating contemplated skin conditions; nevertheless, adding 5,000 units of polymyxin B sulfate and 0.0125% by weight or 3.5 mg/1 oz cream of neomycin sulfate was observed to have afforded a surprisingly effective synergistic combination especially for treating boil and folliculitis. It has been discovered prerequisite antibiotic functionality contemplated herein has been achieved by any of the following active ingredients either in singular dose or in a combination within respective specified dosage concentration ranges established for each ingredient—in the specified dosage form thereof: (a) bacitracin ointment containing, in each gram, 500 units of bacitracin in a suitable ointment base; (b) bacitracin zinc ointment containing, in each gram, 500 units of bacitracin zinc in a suitable ointment base; (c) chlortetracycline hydrochloride ointment containing, in each gram, 30 milligrams of chlortetracycline hydrochloride in a suitable ointment base; (d) neomycin sulfate ointment containing, in each gram, 3.5 milligrams of neomycin in a suitable water soluble or oleaginous ointment base; (e) neomycin sulfate cream containing, in each gram, 3.5 milligrams of neomycin in a suitable cream base; (f) tetracycline hydrochloride ointment containing, in each gram, 30 milligrams of tetracycline hydrochloride in a suitable ointment base; and mixtures thereof.
Moreover, antibiotic functionality prerequisite for topical OTC formulations contemplated hereunder have been achieved by conventionally blending combinations of particular active ingredients provided each active ingredient is present within each of the following established concentration and in the concomitant specified dosage form:

(a) Combinations of Antibiotic Active Ingredients:
(1) bacitracin-neomycin sulfate ointment containing per gram 500 units of bacitracin and 3.5 milligrams of neomycin in a suitable ointment base; (2) bacitracin-neomycin sulfate-polymyxin B sulfate ointment containing per gram—in a suitable ointment base—(i) 500 units of bacitracin, 3.5 milligrams of neomycin, and 5,000 units of polymyxin B; or (ii) 400 units of bacitracin, 3.5 milligrams of neomycin, and 5,000 units of polymyxin B; (3) bacitracin-polymyxin B sulfate topical aerosol containing per gram 500 units of bacitracin and 5,000 units of polymyxin B in a suitable vehicle, packaged in a pressurized container with suitable inert gases; (4) bacitracin zinc-neomycin sulfate ointment containing per gram 500 units of bacitracin and 3.5 milligrams of neomycin—in a suitable ointment base; (5) bacitracin zinc-neomycin sulfate-polymyxin B sulfate ointment containing per gram—in a suitable ointment base: (i) 400 units of bacitracin, 3 milligrams of neomycin, and 8,000 units of polymyxin B; or (ii) 400 units of bacitracin, 3.5 milligrams of neomycin, and 5,000 units of polymyxin B; or (iii) 500 units of bacitracin, 3.5 milligrams of neomycin, and 5,000 units of polymyxin B; or (iv) 500 units of bacitracin, 3.5 milligrams of neomycin, and 10,000 units of polymyxin B;
(6) bacitracin zinc-polymyxin B sulfate ointment containing, in each gram, 500 units of bacitracin and 10,000 units of polymyxin B in a suitable ointment base.
(7) bacitracin zinc-polymyxin B sulfate topical aerosol containing, in each gram, 120 units of bacitracin and 2,350 units of polymyxin B in a suitable vehicle, packaged in a pressurized container with suitable inert gases.
(8) bacitracin zinc-polymyxin B sulfate topical powder containing, in each gram, 500 units of bacitracin and 10,000 units of polymyxin B in a suitable base.
(9) neomycin sulfate-polymyxin B sulfate ointment containing, in each gram, 3.5 milligrams of neomycin and 5,000 units of polymyxin B in a suitable water miscible base.
(10) Neomycin sulfate-polymyxin B sulfate cream containing, in each gram, 3.5 milligrams of neomycin and 10,000 units of polymyxin B in a suitable vehicle.
(11) Oxytetracycline hydrochloride-polymyxin B sulfate ointment containing, in each gram, 30 milligrams of oxytetracycline and 10,000 units of polymyxin B in a suitable ointment base.
(12) Oxytetracycline hydrochloride-polymyxin B sulfate topical powder containing, in each gram, 30 milligrams of oxytetracycline and 10,000 units of polymyxin B with a suitable filler.

(b) Combinations of First Aid Antibiotic Active Ingredients and Local Anesthetic Active Ingredients:
(1) bacitracin ointment containing, in each gram, 500 units of bacitracin and any single generally recognized as safe and effective amine or “caine”-type local anesthetic active ingredient in a suitable ointment base.
(2) bacitracin-neomycin sulfate-polymyxin B sulfate ointment containing, in each gram, in a suitable ointment base the following:
(i) 500 units of bacitracin, 3.5 milligrams of neomycin, 5,000 units of polymyxin B, and any single generally recognized as safe and effective amine or “caine”-type local anesthetic active ingredient; or
(ii) 400 units of bacitracin, 3.5 milligrams of neomycin, 5,000 units of polymyxin B, and any single generally recognized as safe and effective amine or “caine”-type local anesthetic active ingredient.
(3) bacitracin-polymyxin B sulfate topical aerosol containing per gram 500 units of bacitracin and 5,000 units of polymyxin B and any single generally recognized as safe and effective amine or “caine”-type local anesthetic active ingredient—in a suitable vehicle—packaged in a pressurized container with suitable inert gases.
(4) bacitracin zinc-neomycin sulfate-polymyxin B sulfate ointment containing per gram—in a suitable ointment base: (i) 400 units of bacitracin, 3 milligrams of neomycin, 8,000 units of polymyxin B, and any single generally recognized as safe and effective amine or “caine”-type local anesthetic active ingredient; or (ii) 400 units of bacitracin, 3.5 milligrams of neomycin, 5,000 units of polymyxin B, and any single generally recognized as safe and effective amine or “caine”-type local anesthetic active ingredient; or (iii) 500 units of bacitracin, 3.5 milligrams of neomycin, 5,000 units of polymyxin B, and any single generally recognized as safe and effective amine or “caine”-type local anesthetic active ingredient; or (iv) 500 units of bacitracin, 3.5 milligrams of neomycin, 10,000 units of polymyxin B, and any single generally recognized as safe and effective amine or “caine”-type local anesthetic active ingredient;
(5) Bacitracin zinc-polymyxin B sulfate ointment containing per gram 500 units of bacitracin, 10,000 units of polymyxin B, and any single generally recognized as safe and effective amine or “caine”-type local anesthetic active ingredient—in a suitable ointment base.
(6) Neomycin sulfate-polymyxin B sulfate cream containing per gram 3.5 milligrams of neomycin, 10,000 units of polymyxin B, and any single generally recognized as safe and effective amine or “caine”-type local anesthetic active ingredient—in a suitable vehicle; and mixtures thereof.

### Anesthetic Ingredients

Anesthetic ingredients have been found to be needed to be mixed with other active and inactive ingredients to provide an effective composition suitable for treating certain painful papule-like boil or ingrown hair conditions. Pramoxine 1% by weight was found to be effective for relieving such pain; similarly, lidocaine 1% by weight was also found to be effective when combined with a vasoconstrictor agent ingredient taught hereunder. Surprisingly, it has been ascertained anesthetic dosage generally may be increased by a factor of 4, i.e., from 1% to as much as 4% without significantly deviating from the skin treatment objectives articulated herein. Importantly, any of the hereinbefore recited vasoconstrictor ingredients—especially when being dissolved in solution in combination with lidocaine—is not recommended for treatment implicating affected skin proximal to areas inherently characterized by limited blood supply, namely, the digits, penis, tip of the nose, and earlobe on the basis of being susceptible to ischemia and necrosis. Accordingly, acceptable local anesthetic active ingredient combinations have been found to fall within the following established dosage concentrations and in the specified dosage form: (a) benzocaine 5 to 20 percent; (b) benzyl alcohol 1 to 4 percent; (c) dibucaine 0.25 to 1 percent; (d) dibucaine hydrochloride 0.25 to 1 percent; (e) dyclonine hydrochloride 0.5 to 1 percent; (f) lidocaine 2 to 5 percent; (g) pramoxine hydrochloride 1 percent; (h) tetracaine 0.5 to 1 percent; (i) tetracaine hydrochloride 0.5 to 1 percent; and mixtures thereof.

### Analgesia Ingredients

Analgesia Ingredients are well known in the art as being commonly blended into medications to relieve pain without concomitant loss of feeling. For example, a Nonsteroidal Anti-Inflammatory drug (NSAID) such as topical gel has recently been accorded FDA approval as an OTC ingredient. Accordingly, 1% diclofenac sodium may be added to OTC compositions for reducing mild pain typically associated with acne, pustules or other similar abnormal skin conditions; an effective range has been found to be 1% to 2% diclofenac sodium topical gel as subsumed into the following common products within a specific dosage concentration range: (a) Camphor 0.1 to 3 percent; (b) Juniper tar 1 to 5 percent; (c) Menthol 0.1 to 1 percent; and mixtures thereof.

### Antifungal Ingredients

Antifungal ingredients seemingly unavoidably tend to increase likelihood of engendering fungal and yeast overgrowth or related infection as adverse side-effects associated with simultaneously applying antibiotic ingredients to treat skin conditions, since such antibiotic ingredients inherently tend to kill good bacteria sustaining skin's micro flora balance. Accordingly, to address this challenging and potentially counterproductive issue, clotrimazole 1% by weight has been mixed into formulations disclosed herein. Furthermore, it has been found active antifungal ingredients contemplated hereunder preferably consist of any one of the following selections affording prerequisite functionality provided being admixed within the specified concentration range: (a) Clioquinol 3 percent by weight; (b) Haloprogin 1 percent by weight; (c) Miconazole nitrate 2 percent by weight; (d) Povidone-iodine 10 percent by weight; (e) Tolnaftate 1 percent by weight; (f) Undecylenic acid, calcium undecylenate, copper undecylenate, and zinc undecylenate used individually or in any ratio devolving to a total undecylenate concentration of 10 to 25 percent by weight; (g) Clotrimazole 1 percent by weight; and mixtures thereof.

### Secondary, Inactive Ingredients

It has also been ascertained a panoply of secondary or inactive excipient ingredients may advantageously be conventionally blended into topical formulations functioning as carriers, protectants, solvents, suspending agents, and non-ionic surfactants compatible with the plurality of active ingredients contemplated hereunder—either as individual inactive ingredients or in combination to achieve the skin treatment objectives recited hereunder. Such secondary, inactive ingredients may be selected from: (1) Cocoa butter; (2) Glycerin; (3) Hard fat; (4) Mineral oil; (5) Petrolatum; (6) Liquid Petrolatum; (7) White petrolatum; (8) Cottonseed oil; (9) Olive Oil; (10) Sodium pyruvate; (11) Tocopheryl acetate; (12) Eucalyptus Oil; (13) Shea Butter; (14) Cocoa Butter; (15) Vegetable Oil and combinations thereof; (16) Lavender Oil; (17) Eucalyptus Oil; (18) Tea Tree Oil; (19) Glycerin; (20) Purified Water; (21) Acetylated Lanolin Alcohol; (22) Polyoxyethylene Glycol; (23) Glucoside Alkyl Ethers; (24) Sorbitan; and mixtures thereof.

### Efficacious Topical Treatment of Volunteer Subject Patients Skin Conditions

Volunteer Subject Patients have demonstrated significant positive remedial outcomes when subject to treatments relying upon synergistic compositions of antibiotic, vasoconstrictor/decongestant active ingredients, and other secondary ingredients disclosed hereunder and blended together as described herein. Such positive remedial outcomes have been documented by obtaining photographs thereof in real-time and making reference thereto in FIGS. 1-24. As will be hereinafter described in detail in the context of specific adverse skin conditions, this set of drawing figures demonstrate remarkable progress of assuaging symptoms associated with abnormal skin conditions during and subsequent to treatment relative to initial skin conditions.
Skin conditions addressed hereunder are generally characterized as folliculitis or acne. As is known by practitioners in the art, folliculitis is usually associated with an inflammation or infection of hair follicles typically developing into boil characterized by tiny pustules having whiteheads circumscribing individual hair follicles. Folliculitis may also include red skin surrounding hair follicles, implicating itchy, tender, and uncomfortable symptoms, while nevertheless typically not being as painful or deeply penetrating into skin surface as occurs when boil is present.
As is also well known by practitioners in the art, mild acne is usually associated with minor breakouts occurring on facial skin—the cheeks, chin, nose, and forehead. Implicated facial skin inflammation may be characterized as having papules manifest in the form of small pimples or small bumps; as whiteheads manifest in the form of enclosed, plugged pores; and as blackheads manifest in the form of open, plugged pores.

### Volunteer Subject-Patient #1

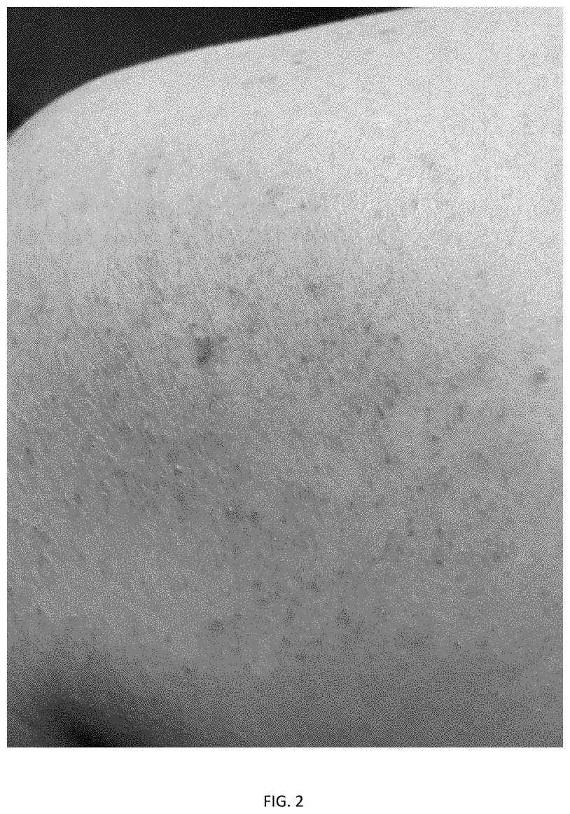

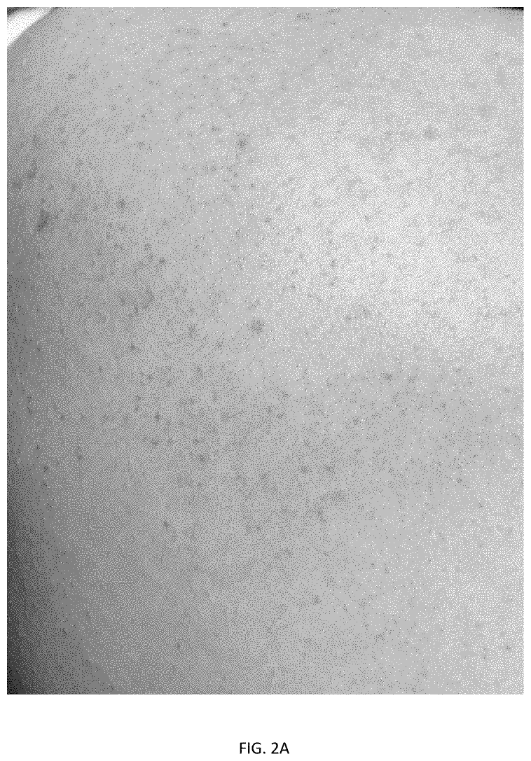

Subject-Patient #1 corresponds to a 20-year-old male having a history of frequently suffering from mild acne on his face and persistent pityrosporum folliculitis on his back associated with chronic red, itchy pustules despite previously receiving multiple doctor-prescribed medical treatments. Referring to FIGS. 2, 2A, 3 and 4, there are depicted frontal views of Subject-Patient #1's upper back and adjacent shoulder area illustrating symptomatic attributes manifest by changes in his implicated skin condition as observed while undergoing a topical treatment regimen via a cream formulation of the instant OTC medication as will be herein described.

More particularly, FIGS. 2 and 2A depict folliculitis condition manifest on upper back and adjacent shoulder prior to commencement of the treatment regimen taught herein. There is seen plurality of small—pinhead-sized—bumps substantially characterized by pimples in the form of papules and pustules located on upper back and situated around adjacent shoulders. Pustules are shown surrounded by inflamed skin and flared skin on upper back and around shoulders. It will be appreciated most pustules are observed as being sized about 1 mm in diameter with an elevation ranging from about 0.5 mm to 1 mm surrounded by inflamed, red skin. Nearby whiteheads appear to be situated in a “popped” condition due to friction with the patient's clothing in contact therewith. A few pustules are noted as being relatively larger than a 1 mm diameter, namely, observed as being about 2-3 mm in diameter. While being treated as elucidated hereinafter, Subject-Patient #1 indicated he had been suffering from this uncomfortable substantially unchecked aberrant skin condition for the last few years—as evidenced by a few deep marks remaining from previously untreated pustules.

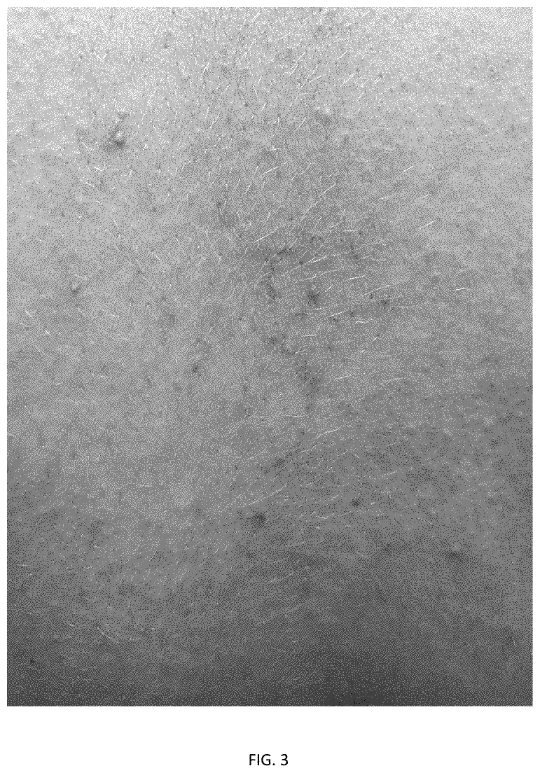

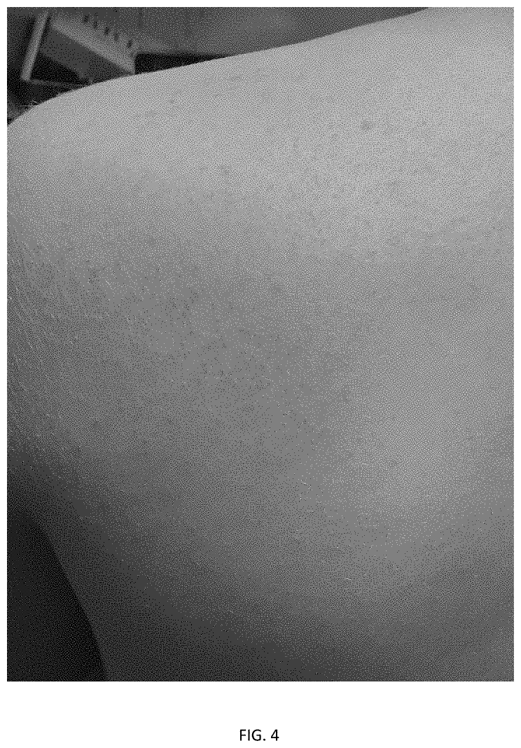

Treatment of Subject-Patient #1's skin condition located on his upper back area and shoulder areas commenced by applying 0.25% by weight of phenylephrine HCl, 500 unit of bacitracin zinc, 5000 unit of polymycin β sulfate, 0.0125% by weight or 3.5 mg/1 oz. of neomycin sulfate, 1% by weight pramoxine and 1% by weight of clotrimazole, via a twice-daily regimen. On the first day after treatment commenced, as illustrated in FIG. 2A, there was a slight reduction in pustules size coupled with surrounding skin becoming less flared. As shown in FIG. 3, on the third day of treatment continued reduction in pustule size and elevation were observed, in addition to being accompanied with reduced skin redness surrounding pustules. Subject-Patient 1's skin condition continued to improve on a daily basis throughout his treatment until six days thereafter wherein, as shown in corresponding frontal view depicted in FIG. 4, the area of implicated skin was gradually and consistently approaching normalcy on the basis of significantly attenuated plurality of pustules and accompanying redness. Nevertheless, indication of previously untreated pustules persisted, albeit manifest by a significantly reduced presence.

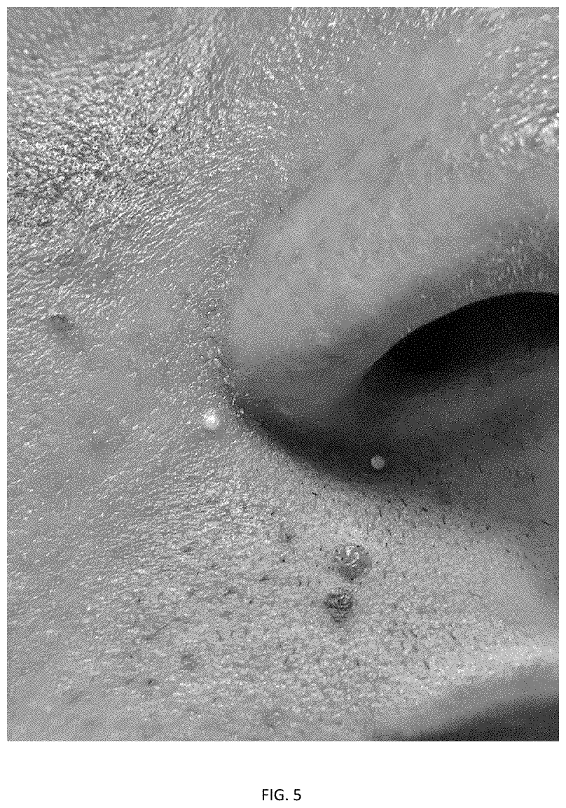

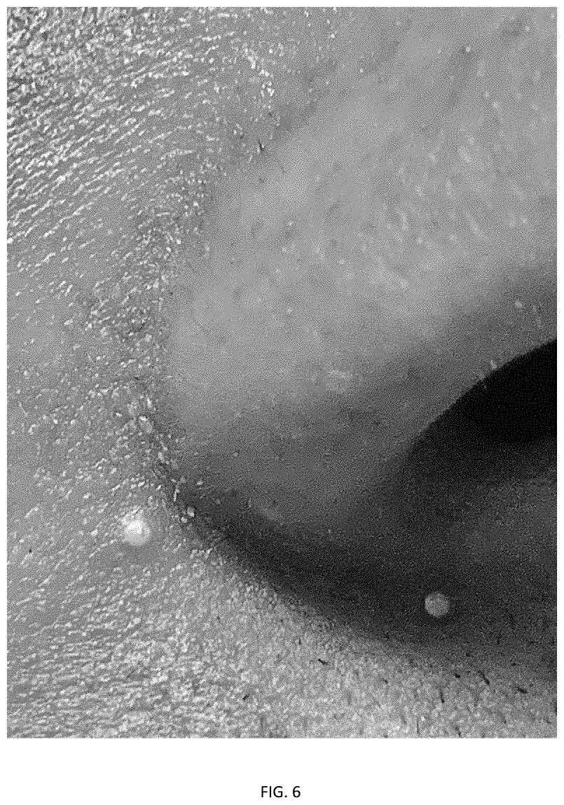

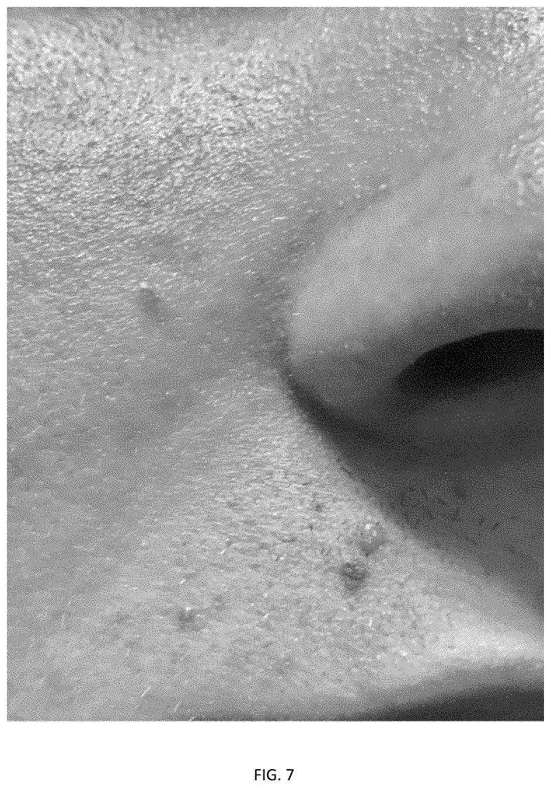

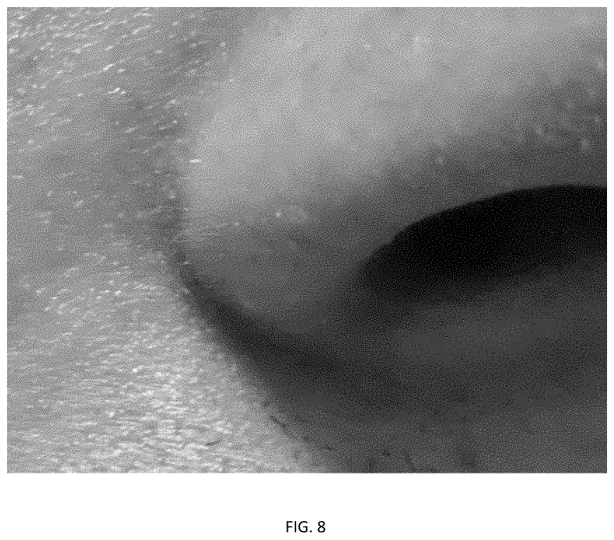

Now referring to FIGS. 5 and 6, there are seen frontal views of Subject-Patient #1's face focusing on an acne condition disposed proximal to his nose nostrils. More particularly, affect upon symptomatic attributes present proximal to his nose nostrils were manifest as plurality of pustules by his acne skin condition were observed while undergoing a topical treatment regimen via a cream formulation of the instant OTC medication as will be herein described. In particular, his facial acne treatment commenced by applying a composition hereof obtained by blending 0.25% by weight of Phenylephrine HCL, 500 unit of bacitracin zinc, 5000 unit of polymycin β sulfate and 0.0125% by weight or 3.5 mg/1 oz. of neomycin sulfate with a plurality of inactive excipient ingredients of sufficient amount to form a cream formulation as contemplated hereunder—via a twice-daily regimen. As depicted in FIG. 7, virtually immediate improvement manifest by abrupt attenuation of pustule size measurements was observed during the first 24 hours of topical treatment. In keeping with this astonishing first-day outcome, as shown in FIG. 8, only two days thereafter infected acne skin area was observed as being devoid of any visible symptoms.

### Volunteer Subject-Patient #2

Subject Patient #2 corresponds to a volunteer 13 year-old teenage girl suffering from a skin condition devolved to a combination of substantial facial acne and bacterial folliculitis. As will be readily appreciated by practitioners in the art, bacterial folliculitis is characterized by itchy, white pus-filled bumps on the skin, i.e., plurality of pustules, typically occurring when hair follicles become infected with bacteria. It is well known in the art that even though hair follicles usually become infected by Staphylococcus aureus bacteria (“staph”) regularly living on a subject's skin, problems also occur when staph enter the subject's body through a cut or other open wound manifest on the skin as pustules. Subject-Patient #2 indicated while her previous treatments based upon doctor-prescribed drugs were effective, prolonged periods of time were prerequisite to effectuate healing of her plurality of facial pustules.

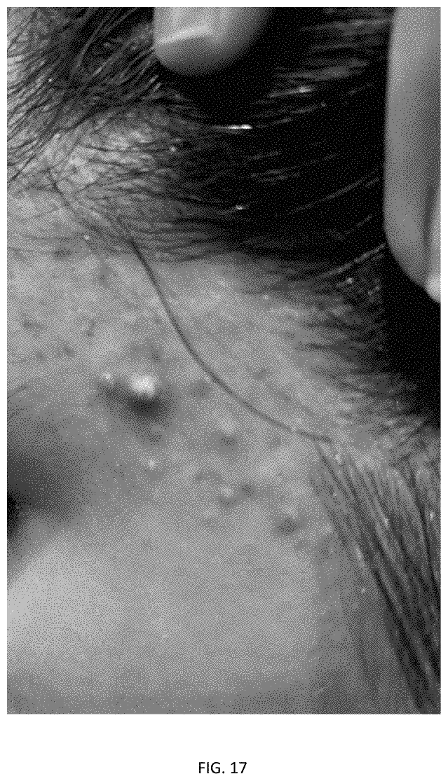

Referring to FIGS. 17-19, there is seen Subject-Patient #2 skin symptomology manifest by the presence of acne characterized by inflamed skin surrounding plurality of largest pustules, about 5 mm diameter, and large pustules, about 2-3 mm diameter, primarily disposed on her temple area interposed between her eye socket and ear. Now specifically focusing on FIG. 17 it was observed that prominent pustules were about 5 mm in diameter and 0.5 mm in elevation and surrounded by red, inflamed and irritated skin. Other nearby pustules appeared to be intermediate size, e.g., about 2-3 mm in diameter, between largest pustules and more regular size pustules, i.e., typically having 1 mm diameter. The patient commented she had been suffering from this adverse, uncomfortable facial skin condition for the past few years evidenced by lingering visible deep marks persisting from untreated pustules.

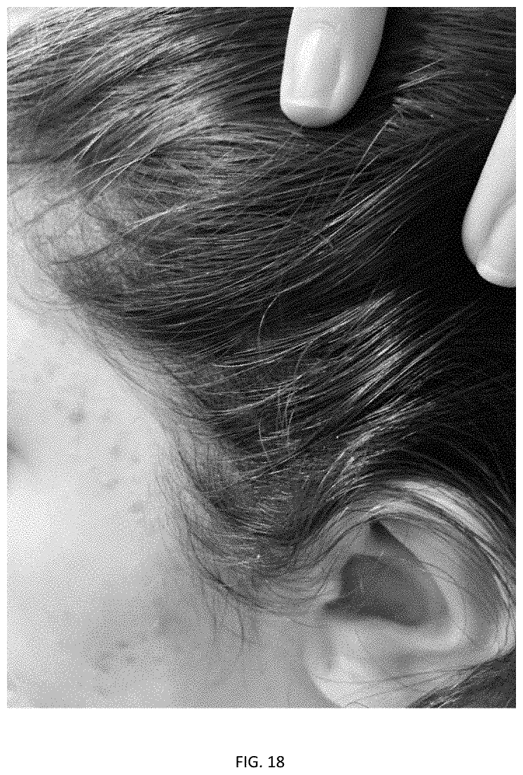

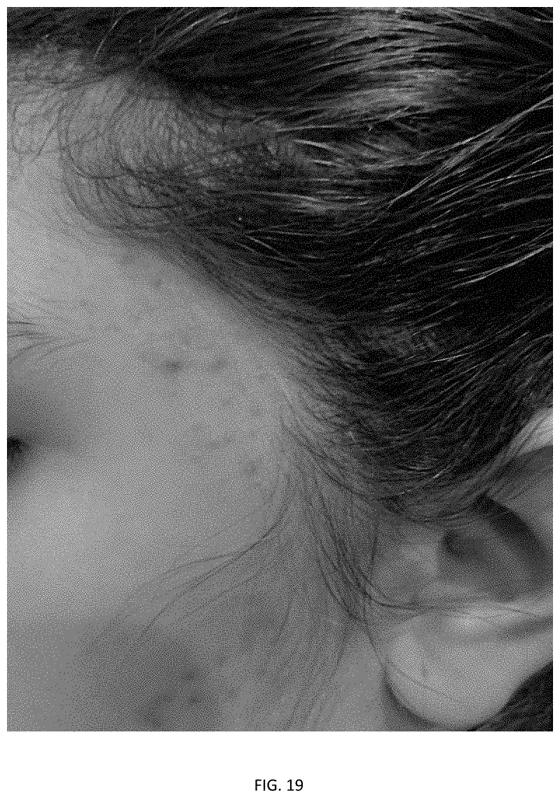

Treatment of Subject-Patient #2 commenced by applying 0.25% by weight of phenylephrine HCl, 500 unit of bacitracin zinc, 5000 unit of polymycin β sulfate, 0.0125% by weight or 3.5 mg/1 oz. of neomycin sulfate, 1% by weight pramoxine and 1% by weight of clotrimazole via a twice daily regimen to address the prevalence of large puss-filled pustules. Nevertheless, in many instances, Subject-Patient #2 seemingly unavoidably failed to properly apply her recommended topical treatment—applying it only once a day on account of frequently forgetting to apply the morning treatment before leaving home to attend school. Significantly, regardless of her failing to rigorously conduct the preferred treatment-schedule, as depicted in FIG. 18, merely one day later, improvement of the implicated acne skin condition was evidenced by only attenuated bumps being visible on her facial skin. Indeed, as further depicted in FIG. 19, after two more day's topical treatment, there was even more pronounced improvement of the targeted facial area manifest by there being a profound paucity of acne-based skin symptoms manifest as being even more attenuated, barely visible bumps.

### Volunteer Subject-Patient #3

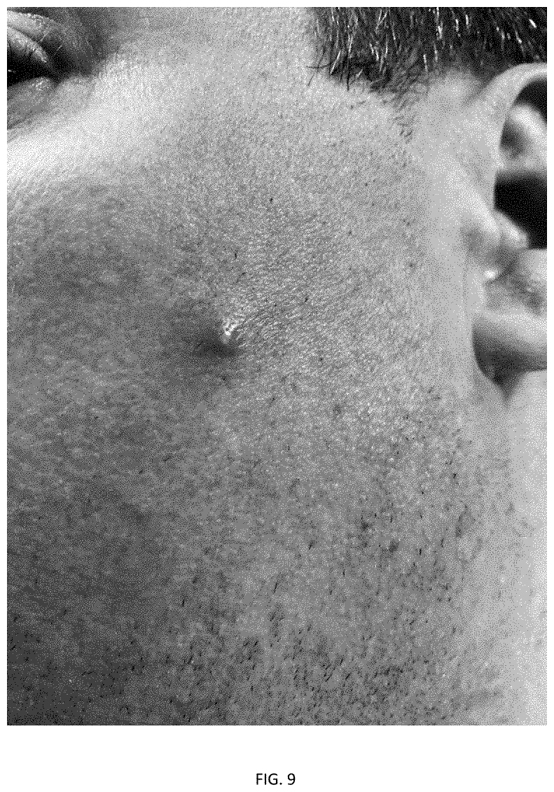

Subject Patient #3 corresponds to a 48-year-old volunteer male who often suffered from boils and folliculitis due to ingrown hair follicles. He also suffered from persistent skin-flaking on and around his ears. On one memorable occasion, as shown in FIGS. 9-12 Subject Patient #3 was stricken by a large and painful pustule on his right cheek. Upon close examination, this condition was identified as being trans-follicular penetration of ingrown hair, wherein hair penetrates hair follicle walls and is caused to grow sideways into surrounding skin. As will be appreciated by practitioners skilled in the art affected hair continues to grow through the epidermal layer of skin. Consequently, areas of trans-follicular penetration typically become inflamed and painful, fraught with pustules situated thereabove. Another aspect of trans-follicular penetration is tendency to be inflicted upon patients having curly and sharp-ended hair, and typically persisted for 3-6 weeks prior to healing.

It is also known that trans-follicular penetration can develop into a boil which is, in turn caused, by staph bacteria routinely carried on the skin or in the noses of many healthy people without any adverse health incident. But, when even an otherwise healthy person suffers a scrape, cut, or splinter implicating skin-penetration, staph bacteria can enter hair follicles and instigate infection. Unfortunately, acne-based boils typically associated with infected clogged pores tend to effectuate significant painful symptoms since the epidermis is fraught with nerve-endings. Accordingly, any attempts at self-help by patients to remove their afflicted hair follicles before reducing or eliminating intertwined swelling of implicated follicles predictably are unsuccessful and very painful. It should be evident to those skilled in the art, heretofore, professional removal of trans-follicular penetration by a dermatologist has been prerequisite to properly and painlessly remove such afflicted hair follicles.

Subject Patient #3 was treated by applying 0.25% by weight of phenylephrine HCl, 500 unit of bacitracin zinc, 5000 unit of polymycin β sulfate, 0.0125% by weight or 3.5 mg/1 oz. of neomycin sulfate and 1% by weight pramoxine, via a twice-daily regimen. The pain was relieved immediately due to the use of pramoxine in the composition functioning as a local anesthetic. Nevertheless, enlarged boil condition remained in conjunction with inflammation surrounding afflicted skin. On the second day of treatment, slight improvement of boil size was observed, i.e., reduced boil diameter and commensurate reduced elevation, coupled with about 50% reduction of inflamed skin area. To cope with relentless resurgence of pain and discomfort abruptly reappearing only a few hours subsequent to treatment seemingly attributable to attenuated effectiveness of local anesthetic incorporated into the formulation, i.e., affording only temporary relief, the treatment regimen was increased from twice to thrice-daily.

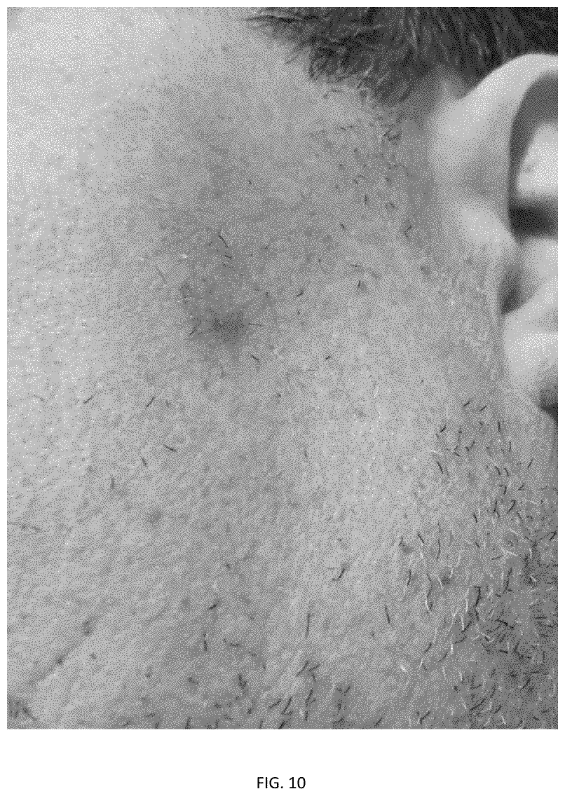

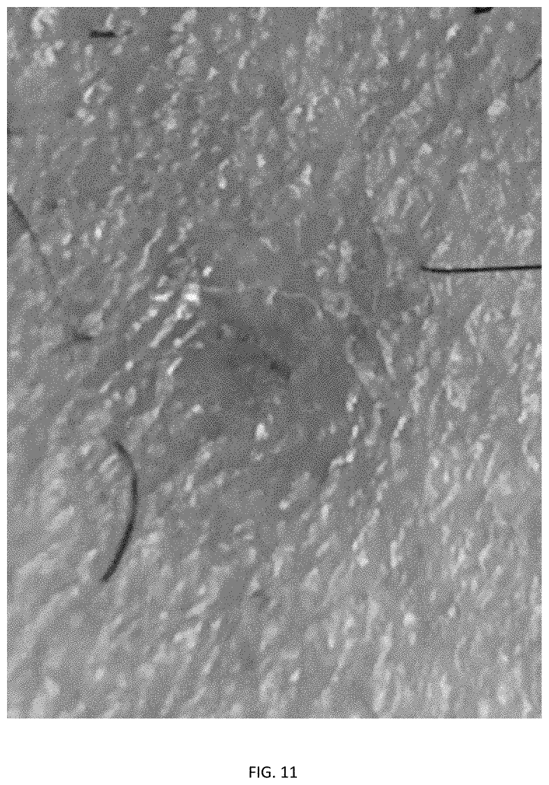

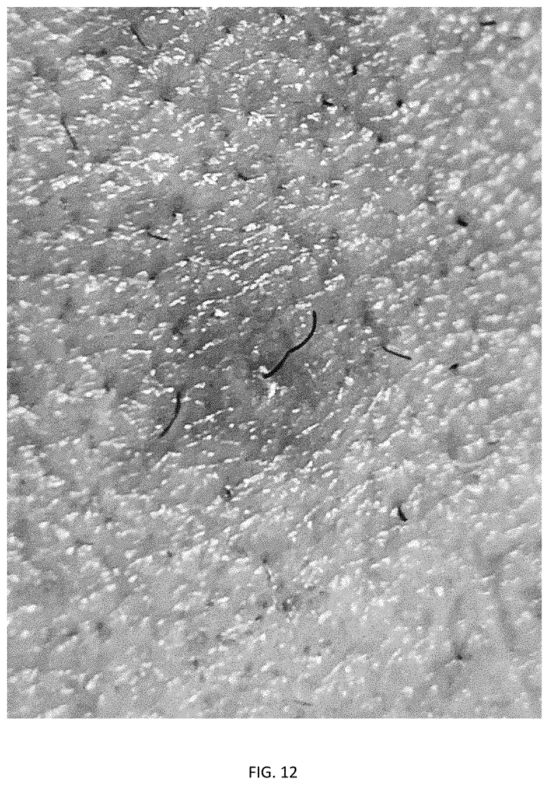

On the basis of this enhanced regimen, as depicted in FIGS. 10-12, consistent daily improvement persisted wherein pustule size and surrounding skin inflammation were gradually being reduced. In particular, after 5 days treatment, FIG. 11 illustrates dramatic swelling reduction wherein boil had been diminished into minor red skin having hair slightly visible from beneath the implicated skin. As this treatment regimen continued for three more days, i.e., until 8 days treatment, as illustrated in FIG. 12, Subject Patient #3's hair was able to be routinely pulled from therebeneath—surprisingly absent any pain or adverse effect.

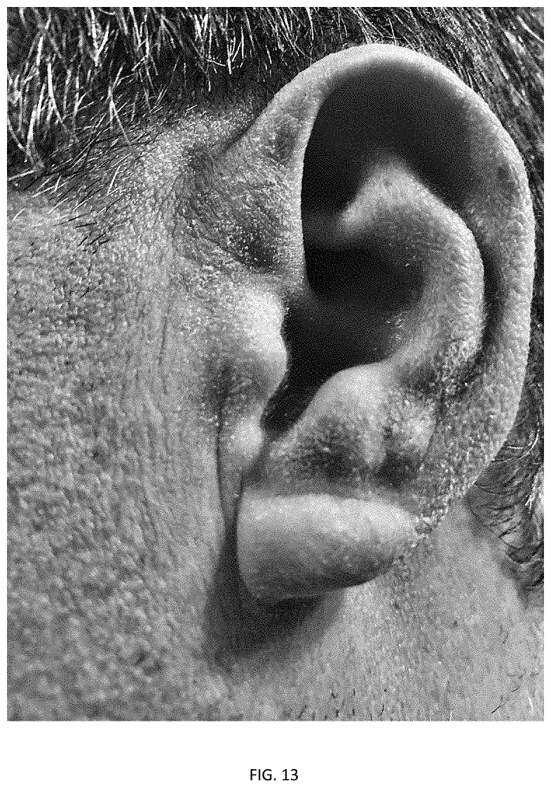

Even though his troublesome boil condition had been significantly attenuated, as depicted in FIG. 13, Subject Patient #3 also suffered from skin-flaking condition on and around his ears. Since skin proximal to ears is inherently regularly exposed to environmental elements such as sun, wind, heat, dryness and excessive humidity, there is clearly high potential for these elements to cause irritation and damage thereto. As should be appreciated by those skilled in the skin treatment art, repeated exposure of uncovered and thus unprotected skin to such environmental elements especially when unavoidably being subjected to extreme conditions, skin-irritation is predictably characterized by significant peeling and concomitant itching. Moreover, it will be appreciated skin-peeling can also result from or be exacerbated by a patient's underlying vulnerability attributable to non-skin-related disease or other condition indirectly triggering an adverse skin side-effect.

A variety of conditions can cause skin-peeling including allergic reactions, staph infections and fungal infections, immune system disorders, cancer and cancer treatments, genetic disease typified by rare skin disorders, e.g., acral-peeling skin syndrome, in turn, causing painless peeling of the outermost epidermal layer. Seeking to personally treat and hopefully remedy his persistent discomfort and suffering from skin-peeling and skin-flaking on and around his ears, Subject-Patient #3 initially applied what he perceived as being an obvious OTC topical choice: skin-moisturizer. But this self-help approach proved to be futile inasmuch as no improvement occurred.

Subject-Patient #3 then opted to receive medical treatment wherein triamcinolone acetonide ointment USP, 0.1% was prescribed on the basis of a twice-daily treatment regimen. During rigorous application of this prescription as instructed, Subject-Patient #3 observed continuously diminished skin-peeling and skin-flaking on and around his ears. This treatment regimen continued resulting in ongoing reduction of skin-peeling and skin-flaking. Nevertheless, shortly thereafter upon discontinuing the twice-daily regimen, skin-flaking promptly returned.

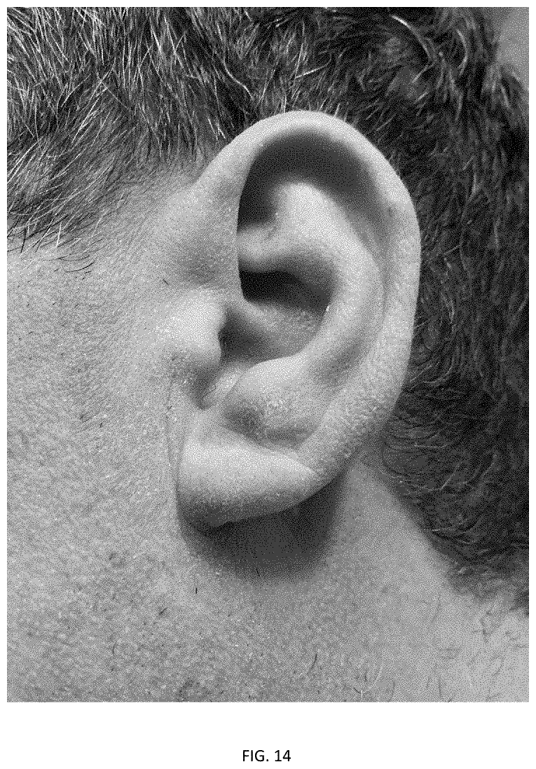

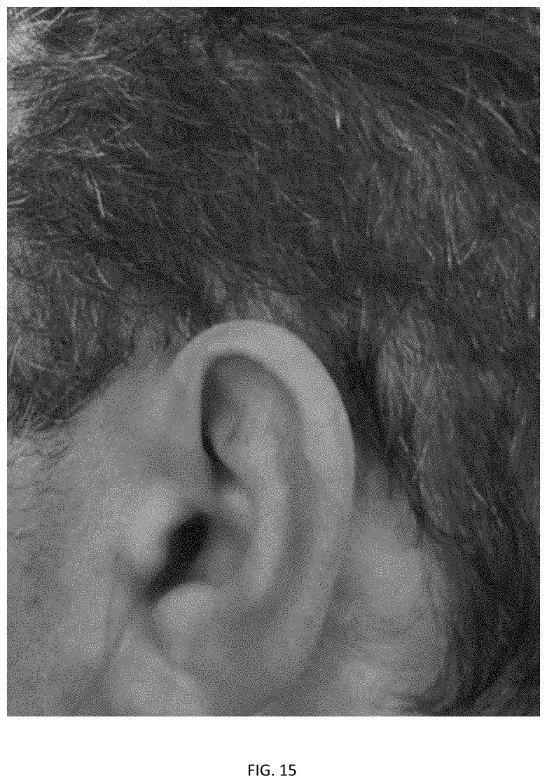

Still referring to the subject-patient's skin-peeling and skin-flaking condition depicted in FIG. 13, he commenced topically applying on a twice-daily basis a combination of 0.25% by weight of phenylephrine HCl, 500 unit of bacitracin zinc, 5000 unit of polymycin β sulfate, 0.0125% by weight or 3.5 mg/1 oz. of neomycin sulfate and 1% by weight pramoxine. As depicted in FIG. 14, substantial improvement was noticed as soon as the first few days thereafter. This successful outcome continued through five days thereafter wherein only very minor skin-flaking was still noticeable on the bottom portion of his ears. Continuing this daily treatment regimen for another four days, effectuated the surprisingly effective outcome depicted in FIG. 15. A normal skin condition was restored on and around the ears, wherein the ears devoid of any visible indication of skin-flaking. In view of his remarkable elimination of ears-related skin-flaking, Subject-Patient #3 discontinued this topical treatment.

There was no recurrence of skin-flaking for a period of more than three months until slight flaking became visible in the lower portion of Subject-Patient #3, seemingly the vulnerable portion thereof. Resumption of the previously efficacious daily treatment regimen applying the hereinbefore specified embodiment of the present invention enabled removal of reappearing skin-flaking after only one week's duration.

### Volunteer Subject-Patient #4

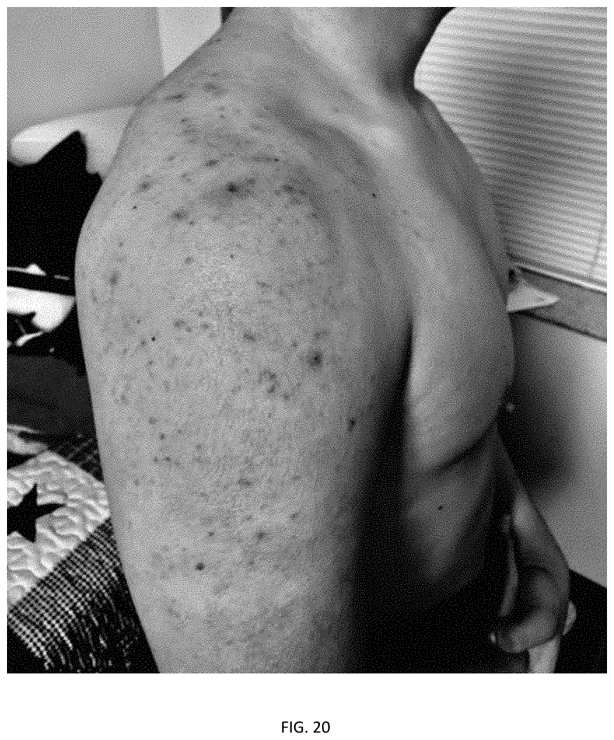

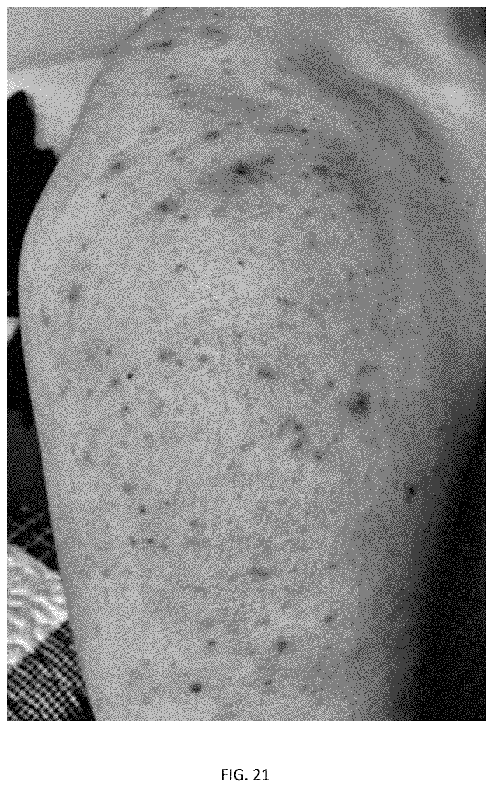

Subject Patient #4 corresponds to a 19 year-old volunteer male who often suffers from persistent folliculitis on his back fraught with pustules circumscribed by inflamed skin concentrated on his upper back and around his shoulders. While this skin condition of was somewhat similar to the skin condition of Subject-Patient #1, there was a significant therebetween: unlike Subject-Patient #1 suffering from a relatively mid skin condition (see FIGS. 2 and 2A), Subject-Patient #4 suffered from a more acute skin condition illustrated in FIGS. 20 and 21; the majority of pustules manifest on Subject-Patient #4's back were about 1-2 mm in diameter and 0.5 mm to 1 mm in elevation surrounded by red skin, with white heads being popped due to frictional contact with his clothes and/or his coping with itching behavior.

A few pustules were observed as being larger than 3 mm diameter surrounded by red, irritated skin. Subject-Patient #4 had been suffering from this adverse condition for a protracted period of time as evidenced by presence of a few visible deep marks indicative of previously untreated pustules. The origin of his folliculitis condition was uncertain, i.e., whether it was attributable to so-called “Hot Tub Folliculitis”—referred to as Pseudomonas Folliculitis—or Pityrosporum Folliculitis or both being associated with rash of red, round, itchy bumps.

As is known in the art, hot tub folliculitis is caused by pseudomonas bacteria which is prevalent in venues including hot tubs and heated pools especially wherein chlorine and pH levels aren't well-regulated. Experientially, normally hot tub folliculitis is contracted one to two days after bacterial exposure, while, contrariwise, pityrosporum folliculitis is chronic and characterized by red, itchy pustules located on a person's back and chest, and sometimes located on the neck, shoulders, upper arms and face too; and is typically caused by a yeast infection.

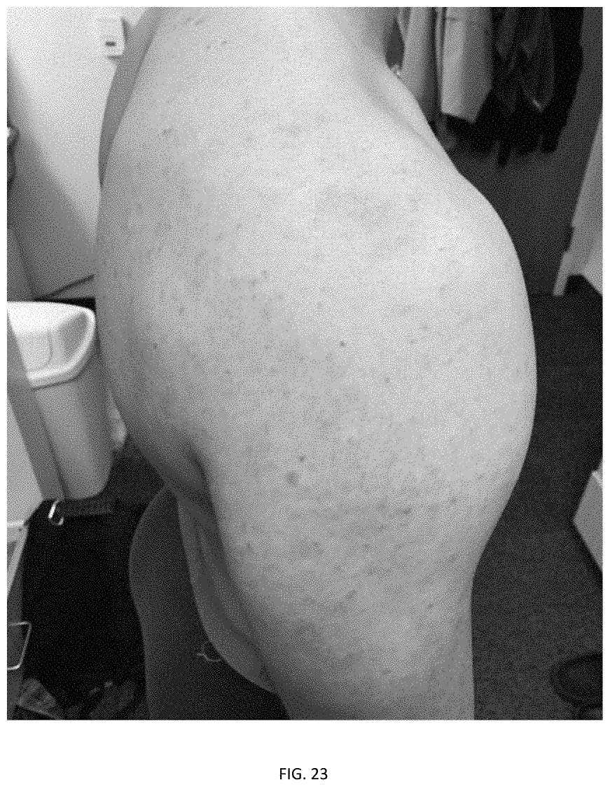

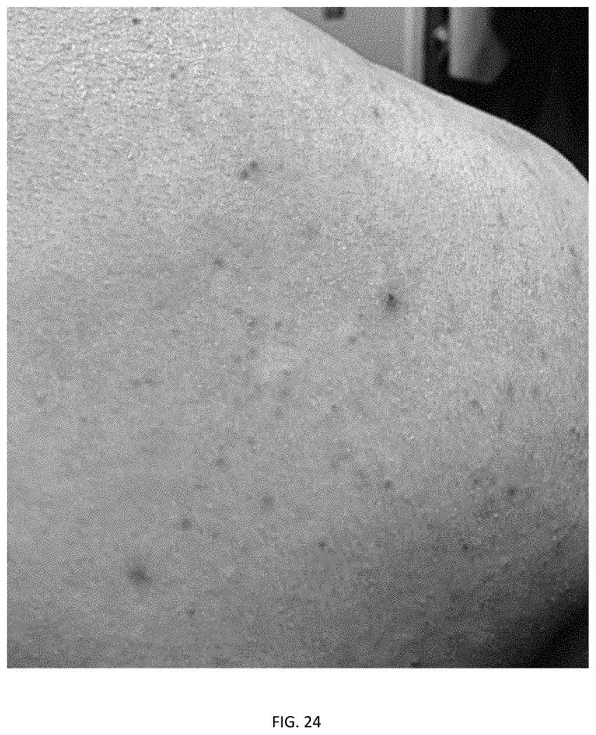

Thus, referring to the severe skin condition depicted in FIGS. 20 and 21, treatment started by applying 0.25% by weight of phenylephrine HCl, 500 unit of bacitracin zinc, 5000 unit of polymycin β sulfate, 0.0125% by weight or 3.5 mg/1 oz. of neomycin sulfate, 1% by weight pramoxine and 1% by weight of Clotrimazole, via a twice-daily regimen. Thereafter, as depicted in FIGS. 23 and 24 improvement was seen on daily basis after initiating the treatment; a slight reduction in pustules size was noticed and the surrounding skin became less flared. On the fourth day of treatment, further reduction of papule size and elevation were observed and the redness circumscribing pustules was likewise attenuated. As depicted specifically in FIG. 24, improvement of skin condition continued through the seventh day wherein the whole area was cleared of pustules and redness. Nevertheless, residual markings associated with previously untreated pustules were still noticeable albeit being reduced.

### Covid-19 Masking Issues

4. COVID-19 mask-covered-area rashes and acne: The current available masks, including textile, surgical, and N95 masks, trap substantial amounts of moisture between the mask and the skin, thereby facilitating a breeding environment for bacteria and fungus. Continuous contact and friction of the skin and mask due to normal mouth movement when speaking or breathing enables bacterial and fungal growth to penetrate the skin and create adverse skin conditions varying from minor rashes to papule growth characterizing acne and folliculitis. These skin conditions were clearly observed in essential service workers at hospitals, restaurants and other venues at which services being rendered require continuous wearing of such masks during the 8-hour work shifts and the like.

It has been noticed that workers subject to the above-mentioned scenarios as well as a plethora of similarly situated scenarios routinely strive to lower their in situ masks, or even to remove the masks completely, particularly as a defensive measure to not only enable their skin to “breathe” normally, but also to remove moisture accumulated on mask-covered skin. Notwithstanding having a righteous purpose, this common worker behavior risks their own health and their customers/client's health by breathing over the items or food that would later be consumed by their peers/customers/clients. Therefore, providing a solution to this problem is very important to aid in containing the spread of COVID-19 virus and other dangerous communicable diseases.

A formulation comprised of 0.25% by weight of phenylephrine HCl, 500 unit of bacitracin zinc, 5000 unit of polymycin β sulfate, 0.0125% by weight or 3.5 mg/1 oz. of neomycin sulfate, 1% by weight pramoxine and 1% by weight of clotrimazole, were tested on various volunteers who suffered from rashes and acne growth under the mask due to their prolonged use thereof. These tests showed tremendous improvement that led to complete healing of their skin conditions in as few as only two days when applying the topical treatment twice daily. It has also been found to be advantageous for even healthy workers and the like to apply the prescribed formulation of combined ingredients once a day to avoid potential bacterial and viral growth and to inherently sustain healthy skin.

### Mosquito Bites Expeditious Remedy

The formulation comprising 0.25% by weight of phenylephrine HCl, 500 unit of bacitracin zinc, 5000 unit of polymycin β sulfate, 0.0125% by weight or 3.5 mg/1 oz. of neomycin sulfate and 1% by weight pramoxine, were proven to be very effective in reducing the swelling and itching of mosquito bites, especially for patients with severe reactions and long-lasting itching sensations, by applying twice daily for three to five days.

This topical treatment for mosquito bites using the compositions taught herein has engendered surprisingly effective virtually instantaneous relief from typically tedious itching. It has been found that even patients having a history of suffering from severe reactions and long-lasting itching from mosquito bites, have assuaged commonly experienced itching in a mere matter of few seconds and furthermore lasted for the rest of the day. Thus, this instantaneous remedial treatment for mosquito bites dwarfed performance of commonly used anti-itch OTC ointments relying upon hydrocortisone 1% as the active ingredient which only reduce insect-bite itching irritation for just a few hours. Based upon these astonishing results it is contemplated that similar remedial results will be achieved for effectively treating bites from fire ants and stings from wasps and bees.

Those skilled in the art will appreciate it has been discovered using synergistic combinations of the active ingredients taught herein, focusing upon surprisingly effective results obtained from incorporation of admixtures of prescribed antibiotic, vasoconstrictor, antifungal, analgesic, and anesthetic active ingredients in conjunction with prescribed excipient secondary ingredients afford especially effective expeditious creams and ointments for topically treating various skin conditions disclosed herein. Moreover, it will be readily appreciated that formulations taught hereunder obtained by blending a plurality of active primary ingredients together with a plurality of inactive secondary ingredients have also been found to be surprisingly effective for treating mosquito bites wherein commonly experienced swelling, redness and itching is attenuated in a matter of mere minutes.

Other variations and modifications will, of course, become apparent from a consideration of the structures and techniques hereinbefore described and depicted. Accordingly, it should be clearly understood that the present invention is not intended to be limited by the particular features and structures hereinbefore described and depicted in the accompanying drawings, but the present invention is to be measured by the scope of the appended claims herein.

What is claimed is:

1. An over-the-counter cream or ointment composition for topically treating skin disorders including acne, folliculitis, rosacea and boil, said cream or ointment composition comprising:
an admixture of active ingredients comprising 0.25% by weight of phenylephrine HCl, 500 unit of bacitracin zinc, 5000 unit of polymycin β sulfate, 0.0125% by weight or 3.5 mg/1 oz. of neomycin sulfate and 1% by weight pramoxine; and
said admixture blended with sufficient inactive excipient ingredients to form said cream or said ointment composition.

2. The over-the-counter cream or ointment composition recited in claim 1, wherein said admixture further comprises: 1% by weight of clotrimazole active ingredient.

3. An over-the-counter cream or ointment composition for topically treating skin disorders including acne, folliculitis, rosacea and boil, said cream or ointment composition comprising:
a first active ingredient selected from the antibiotic group consisting of:
bacitracin ointment containing, in each gram, 500 units of bacitracin; bacitracin zinc ointment containing, in each gram, 500 units of bacitracin zinc;
chlortetracycline hydrochloride ointment containing, in each gram, 30 milligrams of chlortetracycline hydrochloride;
neomycin sulfate ointment containing, in each gram, 3.5 milligrams of neomycin;
neomycin sulfate cream containing, in each gram, 3.5 milligrams of neomycin;
tetracycline hydrochloride ointment containing, in each gram, 30 milligrams of tetracycline hydrochloride;
combination of bacitracin-neomycin sulfate ointment containing per gram 500 units of bacitracin and 3.5 milligrams of neomycin;
combination of bacitracin-neomycin sulfate-polymyxin B sulfate ointment containing per gram—in a suitable ointment base—(i) 500 units of bacitracin, 3.5 milligrams of neomycin, and 5,000 units of polymyxin B; or (ii) 400 units of bacitracin, 3.5 milligrams of neomycin, and 5,000 units of polymyxin B;
combination of bacitracin-polymyxin B sulfate topical aerosol containing per gram 500 units of bacitracin and 5,000 units of polymyxin B;
combination of bacitracin zinc-neomycin sulfate ointment containing per gram 500 units of bacitracin and 3.5 milligrams of neomycin;
combination of bacitracin zinc-neomycin sulfate-polymyxin B sulfate ointment containing per gram: (i) 400 units of bacitracin, 3 milligrams of neomycin, and 8,000 units of polymyxin B; or (ii) 400 units of bacitracin, 3.5 milligrams of neomycin, and 5,000 units of polymyxin B; or (iii) 500 units of bacitracin, 3.5 milligrams of neomycin, and 5,000 units of polymyxin B; or (iv) 500 units of bacitracin, 3.5 milligrams of neomycin, and 10,000 units of polymyxin B;
combination of bacitracin zinc-polymyxin B sulfate ointment containing, in each gram, 500 units of bacitracin and 10,000 units of polymyxin B;
combination of bacitracin zinc-polymyxin B sulfate topical aerosol containing, in each gram, 120 units of bacitracin and 2,350 units of polymyxin B;
combination of bacitracin zinc-polymyxin B sulfate topical powder containing, in each gram, 500 units of bacitracin and 10,000 units of polymyxin B;
combination of neomycin sulfate-polymyxin B sulfate ointment containing, in each gram, 3.5 milligrams of neomycin and 5,000 units of polymyxin B:
combination of neomycin sulfate-polymyxin B sulfate cream containing, in each gram, 3.5 milligrams of neomycin and 10,000 units of polymyxin B;
combination of oxytetracycline hydrochloride-polymyxin B sulfate ointment containing, in each gram, 30 milligrams of oxytetracycline and 10,000 units of polymyxin B; or
combination of oxytetracycline hydrochloride-polymyxin B sulfate topical powder containing, in each gram, 30 milligrams of oxytetracycline and 10,000 units of polymyxin B with a suitable filler;
a second active ingredient selected from the combination of first aid antibiotic active ingredients and local anesthetic active ingredients group consisting of:
bacitracin ointment containing, in each gram, 500 units of bacitracin and any single amine or “caine”-type local anesthetic ingredient;
bacitracin-neomycin sulfate-polymyxin B sulfate ointment containing, in each gram, in a suitable ointment base the following: (i) 500 units of bacitracin, 3.5 milligrams of neomycin, 5,000 units of polymyxin B, and any single amine or “caine”-type local anesthetic active ingredient; or (ii) 400 units of bacitracin, 3.5 milligrams of neomycin, 5,000 units of polymyxin B, and any single amine or “caine”-type local anesthetic active ingredient;
bacitracin-polymyxin B sulfate topical aerosol containing per gram 500 units of bacitracin and 5,000 units of polymyxin B and any single amine or “caine”-type local anesthetic active ingredient;
bacitracin zinc-neomycin sulfate-polymyxin B sulfate ointment containing per gram: (i) 400 units of bacitracin, 3 milligrams of neomycin, 8,000 units of polymyxin B, and any single amine or “caine”-type local anesthetic active ingredient; or (ii) 400 units of bacitracin, 3.5 milligrams of neomycin, 5,000 units of polymyxin B, and any single amine or “caine”-type local anesthetic active ingredient; or (iii) 500 units of bacitracin, 3.5 milligrams of neomycin, 5,000 units of polymyxin B, and any single amine or “caine”-type local anesthetic active ingredient; or (iv) 500 units of bacitracin, 3.5 milligrams of neomycin, 10,000 units of polymyxin B, and any single amine or “caine”-type local anesthetic active ingredient;
bacitracin zinc-polymyxin B sulfate ointment containing per gram 500 units of bacitracin, 10,000 units of polymyxin B, and any single amine or “caine”-type local anesthetic active ingredient; or
neomycin sulfate-polymyxin B sulfate cream containing per gram 3.5 milligrams of neomycin, 10,000 units of polymyxin B, and any single amine or “caine”-type local anesthetic active ingredient;
a third active ingredient selected from the vasoconstrictor group consisting of:
phenylephrine HCl preferably 0.25% by weight with acceptable range of 0.125% to 1.0% by weight;
ephedrine sulfate 0.1 to 1.25 percent;
epinephrine 0.005 to 0.01 percent;
epinephrine hydrochloride 0.005 to 0.01 percent;
phenylephrine hydrochloride 0.25 percent;
phenylephrine hydrochloride 0.125% to 1.0% by weight;
ephedrine sulfate 0.1 to 1.25 percent by weight;
epinephrine 0.005 to 0.01 percent by weight;
epinephrine hydrochloride 0.005 to 0.01 percent by weight; or
phenylephrine hydrochloride 0.25 percent by weight;
a fourth active antifungal ingredient selected from the antifungal group consisting of:
clioquinol 3 percent;
haloprogin 1 percent;
miconazole nitrate 2 percent;
pramoxine hydrochloride 1 percent;
tetracaine 0.5 to 1 percent;
povidone-iodine 10 percent;
tolnaftate 1 percent;
clotrimazole 1 percent; and
a plurality of seventh inactive excipient ingredients selected from the excipient group consisting of cocoa butter; glycerin; mineral oil; liquid petrolatum; white petrolatum; cottonseed oil; sodium pyruvate; tocopheryl acetate; eucalyptus oil; purified water; and sorbitan, wherein each admixture of said first active ingredient, said second active ingredient, said third active ingredient, said fourth active ingredient are blended with sufficient inactive excipient ingredients to from said cream or said ointment composition.

4. The over-the-counter cream or ointment composition for topically treating skin disorders recited in claim 3 further comprising:
a fifth active anesthetic ingredient selected from the anesthetic group consisting of:
benzocaine 5 to 20 percent;
benzyl alcohol 1 to 4 percent;
dibucaine 0.25 to 1 percent;
dyclonine hydrochloride 0.5 to 1 percent; or
lidocaine 2 to 5 percent.

5. The over-the-counter cream or ointment composition for topically treating skin disorders recited in claim 4 further comprising:
a sixth active analgesia ingredients selected from the analgesia group consisting of:
camphor 0.1 to 3 percent;
juniper tar 1 to 5 percent; or
menthol 0.1 to 1 percent.

6. An over-the-counter cream or ointment composition for topically treating skin disorders including acne, folliculitis, rosacea and boil, said cream or ointment composition comprising:
a first active ingredient selected from the antibiotic group consisting of:
bacitracin ointment containing, in each gram, 500 units of bacitracin; bacitracin zinc ointment containing, in each gram, 500 units of bacitracin zinc;
chlortetracycline hydrochloride ointment containing, in each gram, 30 milligrams of chlortetracycline hydrochloride;
neomycin sulfate ointment containing, in each gram, 3.5 milligrams of neomycin;
neomycin sulfate cream containing, in each gram, 3.5 milligrams of neomycin;
tetracycline hydrochloride ointment containing, in each gram, 30 milligrams of tetracycline hydrochloride;
combination of bacitracin-neomycin sulfate ointment containing per gram 500 units of bacitracin and 3.5 milligrams of neomycin;
combination of bacitracin-neomycin sulfate-polymyxin B sulfate ointment containing per gram—in a suitable ointment base—(i) 500 units of bacitracin, 3.5 milligrams of neomycin, and 5,000 units of polymyxin B; or (ii) 400 units of bacitracin, 3.5 milligrams of neomycin, and 5,000 units of polymyxin B;
combination of bacitracin-polymyxin B sulfate topical aerosol containing per gram 500 units of bacitracin and 5,000 units of polymyxin B;
combination of bacitracin zinc-neomycin sulfate ointment containing per gram 500 units of bacitracin and 3.5 milligrams of neomycin;
combination of bacitracin zinc-neomycin sulfate-polymyxin B sulfate ointment containing per gram: (i) 400 units of bacitracin, 3 milligrams of neomycin, and 8,000 units of polymyxin B; or (ii) 400 units of bacitracin, 3.5 milligrams of neomycin, and 5,000 units of polymyxin B; or (iii) 500 units of bacitracin, 3.5 milligrams of neomycin, and 5,000 units of polymyxin B; or (iv) 500 units of bacitracin, 3.5 milligrams of neomycin, and 10,000 units of polymyxin B;
combination of bacitracin zinc-polymyxin B sulfate ointment containing, in each gram, 500 units of bacitracin and 10,000 units of polymyxin B;
combination of bacitracin zinc-polymyxin B sulfate topical aerosol containing, in each gram, 120 units of bacitracin and 2,350 units of polymyxin B;
combination of bacitracin zinc-polymyxin B sulfate topical powder containing, in each gram, 500 units of bacitracin and 10,000 units of polymyxin B;
combination of neomycin sulfate-polymyxin B sulfate ointment containing, in each gram, 3.5 milligrams of neomycin and 5,000 units of polymyxin B:
combination of neomycin sulfate-polymyxin B sulfate cream containing, in each gram, 3.5 milligrams of neomycin and 10,000 units of polymyxin B;
combination of oxytetracycline hydrochloride-polymyxin B sulfate ointment containing, in each gram, 30 milligrams of oxytetracycline and 10,000 units of polymyxin B; or
combination of oxytetracycline hydrochloride-polymyxin B sulfate topical powder containing, in each gram, 30 milligrams of oxytetracycline and 10,000 units of polymyxin B with a suitable filler;
a second active ingredient selected from the combination of first aid antibiotic active ingredients and local anesthetic active ingredients group consisting of:
bacitracin ointment containing, in each gram, 500 units of bacitracin and any single amine or “caine”-type local anesthetic ingredient;
bacitracin-neomycin sulfate-polymyxin B sulfate ointment containing, in each gram, in a suitable ointment base the following: (i) 500 units of bacitracin, 3.5 milligrams of neomycin, 5,000 units of polymyxin B, and any single amine or “caine”-type local anesthetic active ingredient; or (ii) 400 units of bacitracin, 3.5 milligrams of neomycin, 5,000 units of polymyxin B, and any single amine or “caine”-type local anesthetic active ingredient;
bacitracin-polymyxin B sulfate topical aerosol containing per gram 500 units of bacitracin and 5,000 units of polymyxin B and any single amine or “caine”-type local anesthetic active ingredient;
bacitracin zinc-neomycin sulfate-polymyxin B sulfate ointment containing per gram: (i) 400 units of bacitracin, 3 milligrams of neomycin, 8,000 units of polymyxin B, and any single amine or “caine”-type local anesthetic active ingredient; or (ii) 400 units of bacitracin, 3.5 milligrams of neomycin, 5,000 units of polymyxin B, and any single amine or “caine”-type local anesthetic active ingredient; or (iii) 500 units of bacitracin, 3.5 milligrams of neomycin, 5,000 units of polymyxin B, and any single amine or “caine”-type local anesthetic active ingredient; or (iv) 500 units of bacitracin, 3.5 milligrams of neomycin, 10,000 units of polymyxin B, and any single amine or “caine”-type local anesthetic active ingredient;
bacitracin zinc-polymyxin B sulfate ointment containing per gram 500 units of bacitracin, 10,000 units of polymyxin B, and any single amine or “caine”-type local anesthetic active ingredient; or
neomycin sulfate-polymyxin B sulfate cream containing per gram 3.5 milligrams of neomycin, 10,000 units of polymyxin B, and any single amine or “caine”-type local anesthetic active ingredient.
a third active ingredient selected from the vasoconstrictor group consisting of:
phenylephrine HCl preferably 0.25% by weight with acceptable range of 0.125% to 1.0% by weight;
ephedrine sulfate 0.1 to 1.25 percent;
epinephrine 0.005 to 0.01 percent;
epinephrine hydrochloride 0.005 to 0.01 percent;
phenylephrine hydrochloride 0.25 percent;
phenylephrine hydrochloride 0.125% to 1.0% by weight;
ephedrine sulfate 0.1 to 1.25 percent by weight;
epinephrine 0.005 to 0.01 percent by weight;
epinephrine hydrochloride 0.005 to 0.01 percent by weight; or
phenylephrine hydrochloride 0.25 percent by weight;
a fourth active antifungal ingredient selected from the antifungal group consisting of:
clioquinol 3 percent;
haloprogin 1 percent;
miconazole nitrate 2 percent;
pramoxine hydrochloride 1 percent;
tetracaine 0.5 to 1 percent;
povidone-iodine 10 percent;
tolnaftate 1 percent;
clotrimazole 1 percent;
a fifth active anesthetic ingredient selected from the anesthetic group consisting of:
benzocaine 5 to 20 percent;
benzyl alcohol 1 to 4 percent;
dibucaine 0.25 to 1 percent;
dyclonine hydrochloride 0.5 to 1 percent; or
lidocaine 2 to 5 percent;
a sixth active analgesia ingredients selected from the analgesia group consisting of:
camphor 0.1 to 3 percent;
juniper tar 1 to 5 percent; or
menthol 0.1 to 1 percent; and
a plurality of seventh inactive excipient ingredients selected from the excipient group consisting of cocoa butter; glycerin; mineral oil; liquid petrolatum; white petrolatum; cottonseed oil; sodium pyruvate; tocopheryl acetate; eucalyptus oil; purified water; and sorbitan, wherein said admixture of each of said first active ingredient, said second active ingredient, said third active ingredient, said fourth active ingredient, said fifth active ingredient and said sixth active ingredient are blended with sufficient inactive excipient ingredients from said plurality of seventh inactive ingredients to form said cream or said ointment composition.
